(19) United States
(12) Patent Application Publication
(10) Pub. No.: US 2016/0113655 A1
(43) Pub. Date: Apr. 28, 2016

(54) REPOSABLE MULTIPLATFORM ENDOSCOPIC SURGICAL CLIP APPLIER

(71) Applicant: Covidien LP, Mansfield, MA (US)

(72) Inventor: Henry Holsten, Southington, CT (US)

(21) Appl. No.: 14/876,060

(22) Filed: Oct. 6, 2015

Related U.S. Application Data
(60) Provisional application No. 62/068,341, filed on Oct. 24, 2014.

(51) Int. Cl. A61B 17/128

(52) U.S. Cl. CPC A61B 17/1285; A61B 2017/0046

(57) ABSTRACT

A handle assembly for a clip applier includes a housing having a stationary handle and a barrel supported on the stationary handle. The barrel defines a lumen having a proximal portion and a distal portion configured to releasably receive an adapter assembly. A is operably coupled to the housing and includes a gripping portion and a stem portion movable distally through the proximal portion of the lumen in response to movement of the gripping portion towards the stationary handle. A plunger assembly is removably positionable within the proximal portion of the lumen and configured to operably couple the stem portion with an adapter assembly such that distal movement of the stem portion actuates the adapter assembly. A plug is removably engagable within the proximal end of the barrel to retain the stem portion, the plunger assembly, and the adapter assembly in operable relation relative to one another.

### CROSS-REFERENCE TO RELATED APPLICATIONS

This application claims the benefit of and priority to U.S. Provisional Patent Application Ser. No. 62/068,341, filed on Oct. 24, 2014, and incorporated herein by reference in its entirety.

### BACKGROUND

1. Technical Field

The technical field relates to surgical clip appliers. More particularly, the present disclosure relates to a reposable multiplatform endoscopic surgical clip applier having a handle assembly configured for use with a plurality of different adapter assemblies and/or clip cartridge assemblies for firing different types and/or sizes of clips.

2. Description of Related Art

Endoscopic staplers and clip appliers are known in the art and are used for a number of distinct and useful surgical procedures. In the case of a laparoscopic surgical procedure, access to the interior of an abdomen is achieved through narrow tubes or cannulas inserted through a small entrance incision in the skin. Minimally invasive procedures performed elsewhere in the body are often generally referred to as endoscopic procedures. Typically, a tube or cannula device is extended into the patient's body through the entrance incision to provide an access port. The port allows the surgeon to insert a number of different surgical instruments therethrough for performing surgical procedures far removed from the incision.

During a majority of these procedures, the surgeon must often terminate the flow of blood or another fluid through one or more vessels. The surgeon will often apply a surgical clip to a blood vessel or another duct to prevent the flow of body fluids therethrough during the procedure. An endoscopic clip applier is known in the art for applying a single clip during an entry to the body cavity. Such clips are typically fabricated from a biocompatible material and are usually compressed over a vessel. Once applied to the vessel, the compressed clip terminates the flow of fluid therethrough.

Endoscopic clip appliers that are able to apply multiple clips in endoscopic or laparoscopic procedures during a single entry into the body cavity are described in commonly-assigned U.S. Pat. Nos. 5,084,057 and 5,100,420 to Green et al., the contents of which are both incorporated herein by reference in their entirety. Another multiple endoscopic clip applier is disclosed in commonly-assigned U.S. Pat. No. 5,607,436 by Pratt et al., the contents of which are also hereby incorporated by reference herein. These devices are typically, though not necessarily, used during a single surgical procedure. U.S. patent application Ser. No. 08/515,341 now U.S. Pat. No. 5,695,502 to Pier et al., the disclosure of which is hereby incorporated by reference herein in its entirety, discloses a resterilizable surgical clip applier. This resterilizable clip applier is configured to receive and cooperate with an interchangeable clip magazine so as to advance and form multiple clips during a single entry into a body cavity.

During endoscopic or laparoscopic procedures it may be desirable and/or necessary to use different size surgical clips depending on the underlying tissue or vessels to be ligated. In order to reduce overall costs of a surgical clip applier, it is desirable for a single surgical clip applier to be loadable with and capable of firing different size surgical clips as needed.

Accordingly, a need exists for endoscopic surgical clip appliers that include handle assemblies configured for use with different adapter assemblies and/or clip cartridge assemblies for firing different types and/or sizes of clips.

### SUMMARY

Reposable endoscopic surgical clip appliers including handle assemblies configured for use with different adapter assemblies and/or clip cartridge assemblies for firing different types and/or sizes of clips are provided in accordance with the present disclosure. To the extent consistent, any of the aspects or features of the present disclosure may be used in conjunction with any of the other aspects and features of the present disclosure.

A handle assembly for a reposable endoscopic surgical clip applier provided in accordance with the present disclosure includes a housing, a trigger, a plunger assembly, and a plug. The housing includes a stationary handle and a barrel supported on the stationary handle. The barrel includes a proximal end, a distal end, and a longitudinal axis. The barrel further defines a lumen extending between the proximal and distal ends thereof. The lumen is aligned on the longitudinal axis and includes a proximal portion and a distal portion. The distal portion of the lumen is configured to releasably receive an adapter assembly operably associated with a clip cartridge assembly, an end effector, and/or a surgical tool. The trigger is operably coupled to the housing and includes a gripping portion and a stem portion. The gripping portion is movable relative to the stationary handle from an un-actuated position to an actuated position to move the stem portion distally through the proximal portion of the lumen. The plunger assembly is removably positionable within the proximal portion of the lumen and is configured to operably couple the stem portion with an adapter assembly received within the distal portion of the lumen such that distal movement of the stem portion through the proximal portion of the lumen actuates the adapter assembly. The plug is removably engagable within the proximal end of the barrel to close off the proximal portion of the lumen, thereby retaining the stem portion, the plunger assembly, and the adapter assembly in operable relation relative to one another.

In aspects of the present disclosure, the plunger assembly includes a plunger having a head and a shaft extending distally from the head. In such aspects, upon distal movement of the stem portion through the proximal portion of the lumen, the stem portion is urged into contact with the head to thereby urge the plunger distally to actuate the adapter assembly.

In aspects of the present disclosure, the plunger assembly further includes a flange slidably disposed about the shaft and a biasing member disposed about the shaft and positioned between the flange and the head. In such aspects, the flange may be configured to abut a shoulder defined within the proximal portion of the lumen when the plunger assembly is positioned therein such that the biasing member is compressed upon distal movement of the plunger through the proximal portion of the lumen.

In aspects of the present disclosure, the plug is configured for threaded engagement within the proximal end of the barrel.

In aspects of the present disclosure, a lock collar is provided. The lock collar is rotatably coupled to the barrel and extends distally therefrom. The lock collar is rotatable relative to both the barrel and the adapter assembly to releasably engage the adapter assembly within the distal portion of the lumen.

In aspects of the present disclosure, the housing, the trigger, the plunger assembly, and the plug are configured as resterilizable, reusable components.

A reposable endoscopic surgical clip applier provided in accordance with aspects of the present disclosure includes a handle assembly and a plurality of adapter assemblies. The handle assembly includes a housing, a trigger, a plunger assembly, and a plug. The housing includes a stationary handle and a barrel supported on the stationary handle. The barrel includes a proximal end, a distal end, and a longitudinal axis. The barrel defines a lumen extending between the proximal and distal ends thereof. The lumen is aligned on the longitudinal axis and includes a proximal portion and a distal portion. The trigger is operably coupled to the housing and movable relative to the stationary handle from an un-actuated position to an actuated position. The plunger assembly is removably positionable within the proximal portion of the lumen and is configured to operably couple to the trigger such that movement of the trigger from the un-actuated position to the actuated position advances the plunger assembly distally though the proximal portion of the lumen. The plug is removably engagable within the proximal end of the barrel to close off the proximal portion of the lumen, thereby retaining the trigger in operable relation relative to the plunger assembly.

Each of the adapter assemblies is operably associated with a clip cartridge assembly, an end effector, and/or a surgical tool and is configured for releasable receipt with the distal portion of the lumen in operable relation relative to the plunger assembly such that distal movement of the plunger assembly through the proximal portion of the lumen actuates the clip cartridge assembly, end effector, or surgical tool associated with that adapter assembly. At least one adapter assembly of the plurality of adapter assemblies has a stroke length which is different from a stroke length of the remaining adapter assemblies.

In aspects of the present disclosure, the trigger includes a gripping portion and a stem portion. The gripping portion of the trigger is movable relative to the stationary handle from the un-actuated position to the actuated position to move the stem portion distally through the proximal portion of the lumen to advance the plunger assembly distally though the proximal portion of the lumen.

In aspects of the present disclosure, the plunger assembly includes a plunger having a head and a shaft extending distally from the head. Upon distal movement of the stem portion through the proximal portion of the lumen, the stem portion is urged into contact with the head to thereby urge the plunger distally.

In aspects of the present disclosure, the plunger assembly further includes a flange slidably disposed about the shaft and a biasing member disposed about the shaft and positioned between the flange and the head. In such aspects, the flange may be configured to abut a shoulder defined within the proximal portion of the lumen when the plunger assembly is positioned therein such that the biasing member is compressed upon distal movement of the plunger through the proximal portion of the lumen.

In aspects of the present disclosure, the handle assembly further includes a lock collar rotatably coupled to the barrel and extending distally therefrom. With one of the adapter assemblies inserted into the distal portion of the lumen, the lock collar is rotatable relative to the barrel and that adapter assembly to releasably engage that adapter assembly within the distal portion of the lumen.

In aspects of the present disclosure, one or more of the adapter assemblies includes an outer shell, a collar slidably disposed within the outer shell, a shaft engaged with the collar and extending distally from the collar and the outer shell, and a plunger assembly slidably disposed within the outer shell. In such aspects, the plunger assembly of the adapter assembly may include a stem and a plunger slidable relative to the stem and including a head and a shaft extending distally from the head. Further still, in such aspects, the plunger assembly may include a biasing member disposed about the stem and the shaft of the plunger and positioned between the collar and the head of the plunger such that slidable insertion of the shaft of the plunger into the stem compresses the biasing member.

In aspects of the present disclosure, distal movement of the plunger of the handle assembly urges the plunger of the adapter assembly distally to actuate the clip cartridge assembly, end effector, or surgical tool associated with that adapter assembly.

A method of assembling a reposable endoscopic surgical clip applier provided in accordance with aspects of the present disclosure includes inserting a plunger assembly into a proximal portion of a lumen defined within a barrel of a housing. The housing further includes a stationary handle supporting the barrel thereon. The method further includes engaging a plug within the proximal portion of the lumen of the barrel to close off the proximal portion of the lumen of the barrel and retain the plunger assembly therein, inserting an adapter assembly into a distal portion of the lumen of the barrel such that the adapter assembly is operably positioned relative to the plunger assembly, and engaging the adapter assembly within the distal portion of the lumen of the barrel.

In aspects of the present disclosure, the method further includes, prior to engaging the plug, moving a gripping portion of a trigger operably associated with the housing towards the stationary handle such that a stem portion of the trigger abuts a proximal end of the plunger assembly.

In aspects of the present disclosure, engaging the adapter assembly within the distal portion of the lumen of the barrel includes rotating a lock collar relative to both the adapter assembly and the barrel.

### BRIEF DESCRIPTION OF THE DRAWINGS

Aspects and features of the present disclosure are described herein with reference to the accompanying drawings, wherein:

FIG. 1 is an exploded, perspective view of a handle assembly of a reposable endoscopic surgical clip applier provided in accordance with aspects of the present disclosure;

FIG. 2 is a partial longitudinal, cross-sectional view of the handle assembly of FIG. 1, with a first adapter assembly operably engaged therein;

FIG. 3 is a perspective view of a proximal end of the first adapter assembly of FIG. 2 and a proximal end of a second adapter assembly provided in accordance with aspects of the present disclosure and configured for use with the handle assembly of FIG. 1;

FIG. 4 is a perspective view of the proximal end of the first adapter assembly of FIG. 2 with the outer tubular shell removed to illustrate the internal components thereof;

FIG. 5 is a perspective view of the proximal end of the first adapter assembly of FIG. 2 with a portion of the outer tubular shell removed to illustrate the internal components thereof; and

FIG. 6 is a perspective view of the second adapter assembly of FIG. 3 illustrating the tubular shaft thereof extending distally from the outer tubular shell thereof.

### DETAILED DESCRIPTION OF EMBODIMENTS

Embodiments of reposable endoscopic surgical clip appliers provided in accordance with the present disclosure will now be described in detail with reference to the drawing figures wherein like reference numerals identify similar or identical structural elements. As shown in the drawings and described throughout the following description, as is traditional when referring to relative positioning on a surgical instrument, the term “proximal” refers to the end of the apparatus or component thereof which is closer to the user and the term “distal” refers to the end of the apparatus or component thereof which is further from the user.

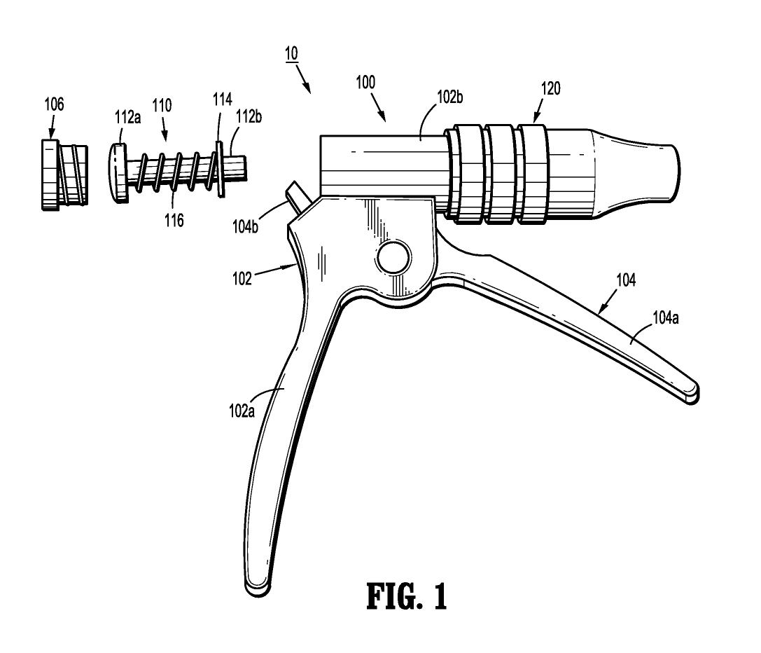

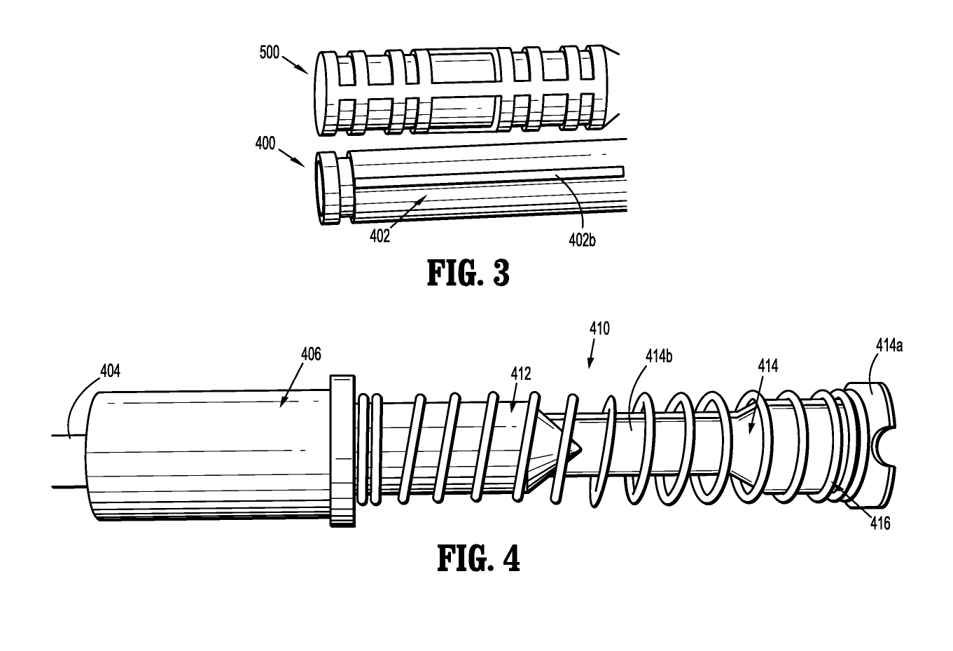

Turning to FIGS. 1-3, an endoscopic surgical clip applier provided in accordance with the present disclosure is generally designated as 10. Surgical clip applier 10 generally includes a handle assembly 100 and a plurality of adapter assemblies, e.g., adapter assemblies 400, 500 (FIG. 3), configured for releasable engagement and use with handle assembly 100. Each adapter assembly 400, 500 is configured for actuating one or more particular clip cartridge assemblies (not shown), end effectors (not shown), or surgical tools (not shown). The adapter assemblies 400, 500 may be reusable components configured for releasable engagement and use with one or more disposable clip cartridge assemblies, disposable/reusable end effectors, and/or disposable/reusable surgical tools. Alternatively, the adapter assemblies 400, 500 may be integral with corresponding clip cartridge assemblies, end effectors, or surgical tools and, together, may be reusable or disposable. Handle assembly 100 is configured as a reusable component.

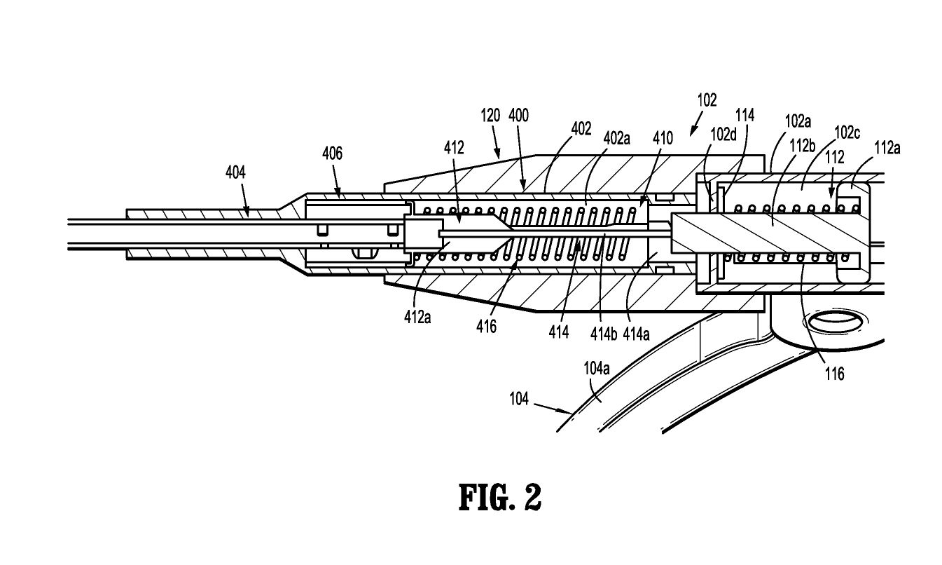

With reference to FIG. 1, handle assembly 100 of surgical clip applier 10 includes a housing 102, a trigger 104, a plug 106, and a plunger assembly 110. Housing 102 includes a stationary handle portion 102a, and a barrel portion 102b supported on stationary handle portion 102a. Barrel portion 102b defines a lumen 102c (FIG. 2) therethrough. In some embodiments, barrel portion 102b includes a lock collar 120 rotatably coupled to barrel portion 102b and extending distally therefrom. Housing 102 of handle assembly 100 may be formed of a suitable plastic or thermoplastic material, or from stainless steel or the like.

Trigger 104 of handle assembly 100 is pivotably connected to stationary handle portion 102a of housing 102. Trigger 104 includes a finger grip portion 104a projecting from stationary handle portion 102a and/or barrel portion 102b of housing 102, and an actuation stem 104b configured to move into lumen 102c (FIG. 2) of barrel portion 102b of housing 102, as detailed below. Trigger 104 may be formed of a suitable plastic or thermoplastic material, or from stainless steel or the like.

Plug 106 of handle assembly 100 is releasably securable within a proximal end of barrel portion 102b of housing 102 for closing the proximal end of barrel portion 102b and lumen 102c (FIG. 2) of housing 102. Plug 106 may be connectable to barrel portion 102b of housing 102 via screw threads, a bayonet-type arrangement, or other suitable releasable engagement structure. Plug 106 may be formed of a suitable plastic or thermoplastic material, or from stainless steel or the like.

Plunger assembly 110 of handle assembly 100 is removably supported within lumen 102c (FIG. 2) of barrel portion 102b of housing 102. Plunger assembly 110 includes a plunger 112 having a head portion 112a and a shaft portion 112b extending distally from head portion 112a. Plunger assembly 110 further includes a flange 114 slidably supported on shaft portion 112b at a location proximal of a distal tip of shaft portion 112b. The distal tip of shaft portion 112b may have an enlarged radial profile, or other suitable feature, thereby preventing flange 114 from being removed from the distal end of shaft portion 112b. Head portion 112a of plunger 112 inhibits flange 114 from being removed from the proximal end of shaft portion 112b. A biasing member 116 of plunger assembly 110 is disposed about shaft portion 112b and interposed between head portion 112a of plunger 112 and flange 114 to bias flange 114 distally relative to shaft portion 112b. The components of plunger assembly 110 may be formed of a suitable plastic or thermoplastic material, or from stainless steel or the like.

With additional reference to FIG. 2, when plunger assembly 110 is loaded into lumen 102c of barrel portion 102b of housing 102, the distal tip of shaft portion 112b of plunger 112 slidably extends through an aperture in a support wall 102d of housing 102, and flange 114 rests adjacent to or in contact with a proximal surface of support wall 102d of housing 102. Following loading of plunger assembly 110 into lumen 102c of barrel portion 102b of housing 102, trigger 104 is pivoted until actuation stem 104b of trigger 104 is moved into lumen 102c of barrel portion 102b adjacent to or in contact with head portion 112a of plunger 112. Plug 106 may then be connected to barrel portion 102b of housing 102 to maintain actuation stem 104b of trigger 104 adjacent to or in operative contact with head portion 112a of plunger 112. At this point, actuation stem 104b is inhibited from returning proximally and is only permitted to move distally upon actuation of trigger 104 towards stationary handle 102a of housing 102.

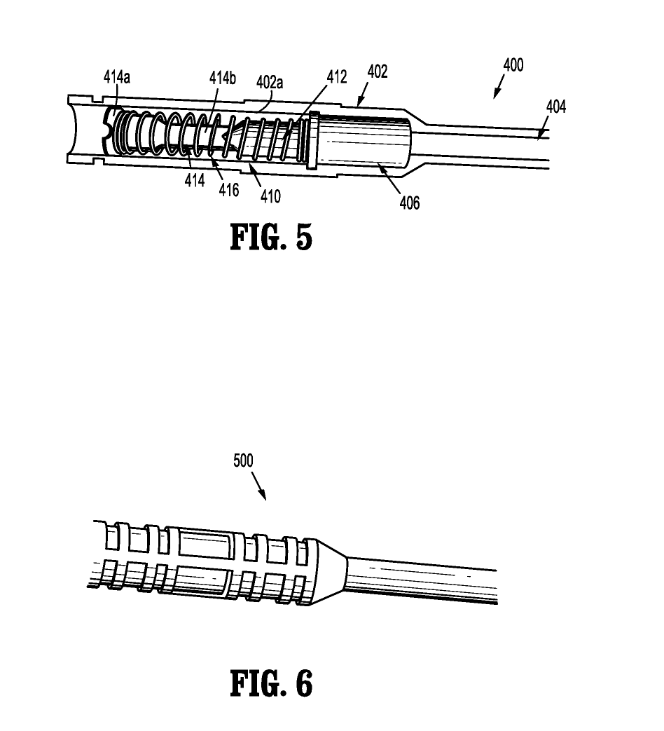

Turning to FIGS. 2-6, as mentioned above, surgical clip applier 10 may include a plurality of adapter assemblies, e.g., a first adapter assembly 400 and a second adapter assembly 500, each configured for operable coupling with handle assembly 100 and interfacing with one or more specific clip cartridge assemblies, end effectors, and/or surgical tools.

With particular reference to FIGS. 2, 4, and 5, first adapter assembly 400 includes an outer tubular shell 402, a tubular shaft 404, and a plunger assembly 410. Shell 402 defines a lumen 402a therethrough. Shell 402 is configured and dimensioned for selective receipt within and connection to a distal portion of lumen 102c of barrel portion 102b of housing 102. Lumen 402a of shell 402 includes a relatively larger diameter proximal portion, and a relatively smaller diameter distal portion.

Shaft 404 of first adapter assembly 400 is fixedly secured to and extends distally from shell 402. Shaft 404 includes a proximal end disposed within the relatively smaller diameter distal portion of shell 402 and extending at least partially into the relatively larger diameter proximal portion of shell 402. A collar 406 is disposed within the relatively larger diameter proximal portion of shell 402 to which the proximal end of shaft 404 is fixedly secured.

Plunger assembly 410 of first adapter assembly 400 is disposed within the relatively larger diameter proximal portion of shell 402. Plunger assembly 410 includes a stem 412 disposed proximally of collar 406. Stem 412 defines a lumen 412a therethrough which is axially aligned with shaft 404. Plunger assembly 410 further includes a plunger 414 having a head portion 414a and a shaft portion 414b extending from head portion 414a. Shaft portion 414b extends into lumen 412a of stem 412 and is slidable relative thereto. Plunger assembly 410 also includes a biasing member 416 disposed about stem 412 of shaft portion 414b of plunger 414 and interposed between head portion 414a of plunger 414 and collar 406 so as to bias plunger 414 proximally relative to collar 406.

First adapter assembly 400 may be connected to handle assembly 100 by a bayonet-type arrangement by inserting shell 402 into the distal portion of lumen 102c of barrel portion 102b of housing 102, and then rotating shell 402 of first adapter assembly 400 relative to barrel portion 102b of housing 102 to engage the thread(s) within the corresponding groove(s) of the bayonet coupling (not shown). Alternatively, first adapter assembly 400 may be connected to handle assembly 100 by inserting shell 402 into the distal portion of lumen or passage 102c of barrel portion 102b of housing 102, and then rotating lock collar 120 of handle assembly 100 relative to both housing 102 and shell 402 to engage the thread(s) with the corresponding groove(s) of the bayonet coupling. Other suitable releasable engagement structures, e.g., mechanical latches, snap-fit connections, etc., are also contemplated.

As best shown in FIG. 3, shell 402 of first adapter assembly 400 may be provided with a longitudinally extending rib 402b. Rib 402b of shell 402 acts like a clocking feature to radially orient and align first adapter assembly 400 with handle assembly 100 for proper connection or coupling of first adapter assembly 400 and handle assembly 100. Handle assembly 100 may include a complementary recess (not shown) or other suitable feature configured to receive rib 402b and align first adapter assembly 400 relative to handle assembly 100. In embodiments where rib 402b is provided, lock collar 120 may also be provided such that lock collar 120 may be rotated relative to both housing 102 and shell 402 (while housing 102 and shell 402 remain stationary relative to one another) to engage first adapter assembly 400 with handle assembly 100, as noted above, thereby maintaining the proper orientation of first adapter assembly 400 relative to handle assembly 100.

Referring to FIGS. 1-5, in operation, with first adapter assembly 400 coupled with handle assembly 100 as detailed above, with plunger assembly 110 loaded in housing 102 as also detailed above, and with a clip cartridge assembly, end effector, or surgical tool connected or coupled to first adapter assembly 400, surgical clip applier 10 is ready for use. In use, as trigger 104 of handle assembly 100 is actuated towards stationary handle portion 102b of housing 102, stem 104b of trigger 104 engages head portion 112a of plunger 112 of plunger assembly 110 and distally advances plunger 112, thereby compressing biasing member 116 between flange 114 and head portion 112a of plunger 112 of plunger assembly 110. As plunger 112 is advanced distally, shaft portion 112b of plunger 112 engages and acts on head portion 414a of plunger assembly 410 of first adapter assembly 400.

With shaft portion 112b of plunger assembly 112 engaging and acting on head portion 414a of plunger 414 of plunger assembly 410 of first adapter assembly 400, plunger 414 is advanced distally, thereby compressing biasing member 416 between collar 406 and head portion 414a of plunger 414. As plunger 414 is advanced in a distal direction, shaft portion 414b of plunger 414 is advanced in a distal direction through lumen 412a of stem 412.

Upon sufficient distal advancement of shaft portion 414b of plunger 414, a distal end of shaft portion 414b of plunger 414 is sufficiently advanced through or beyond lumen 412a of stem 412 so as to act on or otherwise engage the clip cartridge assembly, end effector, or surgical tool operatively connected to first adapter assembly 400 to actuate the respective clip cartridge assembly, end effector, or surgical tool.

Referring to FIGS. 3 and 6, in accordance with the present disclosure, first adapter assembly 400 may be replaced by second adapter assembly 500, or any other suitable adapter assembly having a particular clip cartridge assembly, end effector, or surgical tool operably associated therewith. By replacing first adapter assembly 400 with, for example, second adapter assembly 500, handle assembly 100 (FIG. 1) is capable of actuating the corresponding clip cartridge assembly, end effector, or surgical tool operably associated with second adapter assembly 500, e.g., to actuate different types and/or sizes of clips.

In accordance with the present disclosure, each adapter assembly, for example, adapter assemblies 400 and 500, may have a different or unique stroke length from one another in order to accommodate a particular function of the adapter assembly 400 or 500.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

What is claimed is:

1. A handle assembly for a reposable endoscopic surgical clip applier, the handle assembly comprising:
a housing including a stationary handle and a barrel supported on the stationary handle, the barrel including a proximal end, a distal end, and a longitudinal axis, the barrel defining a lumen extending between the proximal and distal ends thereof, the lumen aligned on the longitudinal axis and including a proximal portion and a distal portion, the distal portion of the lumen configured to releasably receive an adapter assembly operably associated with at least one of: a clip cartridge assembly, an end effector, or a surgical tool;
a trigger operably coupled to the housing and including a gripping portion and a stem portion, the gripping portion movable relative to the stationary handle from an un-actuated position to an actuated position to move the stem portion distally through the proximal portion of the lumen;
a plunger assembly removably positionable within the proximal portion of the lumen, the plunger assembly configured to operably couple the stem portion with an adapter assembly received within the distal portion of the lumen such that distal movement of the stem portion through the proximal portion of the lumen actuates the adapter assembly; and
a plug removably engagable within the proximal end of the barrel to close off the proximal portion of the lumen, thereby retaining the stem portion, the plunger assembly, and the adapter assembly in operable relation relative to one another.

2. The handle assembly according to claim 1, wherein the plunger assembly includes a plunger having a head and a shaft extending distally from the head, and wherein, upon distal movement of the stem portion through the proximal portion of the lumen, the stem portion is urged into contact with the head to thereby urge the plunger distally to actuate the adapter assembly.

3. The handle assembly according to claim 2, wherein the plunger assembly further includes a flange slidably disposed about the shaft and a biasing member disposed about the shaft and positioned between the flange and the head.

4. The handle assembly according to claim 3, wherein the flange is configured to abut a shoulder defined within the proximal portion of the lumen when the plunger assembly is positioned therein such that the biasing member is compressed upon distal movement of the plunger through the proximal portion of the lumen.

5. The handle assembly according to claim 1, wherein the plug is configured for threaded engagement within the proximal end of the barrel.

6. The handle assembly according to claim 1, further including a lock collar rotatably coupled to the barrel and extending distally therefrom, the lock collar rotatable relative to the barrel and the adapter assembly to releasably engage the adapter assembly within the distal portion of the lumen.

7. The handle assembly according to claim 1, wherein the housing, the trigger, the plunger assembly, and the plug are configured as resterilizable, reusable components.

8. A reposable endoscopic surgical clip applier, comprising:
a handle assembly, including:
a housing including a stationary handle and a barrel supported on the stationary handle, the barrel including a proximal end, a distal end, and a longitudinal axis, the barrel defining a lumen extending between the proximal and distal ends thereof, the lumen aligned on the longitudinal axis and including a proximal portion and a distal portion;
a trigger operably coupled to the housing and movable relative to the stationary handle from an un-actuated position to an actuated position;
a plunger assembly removably positionable within the proximal portion of the lumen, the plunger assembly configured to operably couple to the trigger such that movement of the trigger from the un-actuated position to the actuated position advances the plunger assembly distally though the proximal portion of the lumen; and
a plug removably engagable within the proximal end of the barrel to close off the proximal portion of the lumen, thereby retaining the trigger in operable relation relative to the plunger assembly; and
a plurality of adapter assemblies, each of which is operably associated with at least one of: a clip cartridge assembly, an end effector, or a surgical tool, each adapter assembly configured for releasable receipt with the distal portion of the lumen in operable relation relative to the plunger assembly such that distal movement of the plunger assembly through the proximal portion of the lumen actuates the clip cartridge assembly, end effector, or surgical tool associated with that adapter assembly, wherein at least one adapter assembly of the plurality of adapter assemblies has a stroke length which is different from a stroke length of the remaining adapter assemblies.

9. The clip applier according to claim 8, wherein the trigger includes a gripping portion and a stem portion, the gripping portion movable relative to the stationary handle from the un-actuated position to the actuated position to move the stem portion distally through the proximal portion of the lumen to advance the plunger assembly distally though the proximal portion of the lumen.

10. The clip applier according to claim 9, wherein the plunger assembly includes a plunger having a head and a shaft extending distally from the head, and wherein, upon distal movement of the stem portion through the proximal portion of the lumen, the stem portion is urged into contact with the head to thereby urge the plunger distally.

11. The clip applier according to claim 10, wherein the plunger assembly further includes a flange slidably disposed about the shaft and a biasing member disposed about the shaft and positioned between the flange and the head.

12. The clip applier according to claim 11, wherein the flange is configured to abut a shoulder defined within the proximal portion of the lumen when the plunger assembly is positioned therein such that the biasing member is compressed upon distal movement of the plunger through the proximal portion of the lumen.

13. The clip applier according to claim 8, wherein the handle assembly further includes a lock collar rotatably coupled to the barrel and extending distally therefrom, and wherein, with one of the adapter assemblies inserted into the distal portion of the lumen, the lock collar is rotatable relative to the barrel and that adapter assembly to releasably engage that adapter assembly within the distal portion of the lumen.

14. The clip applier according to claim 8, wherein at least one of the adapter assemblies includes:
an outer shell;
a collar slidably disposed within the outer shell;
a shaft engaged with the collar and extending distally from the collar and the outer shell; and
a plunger assembly slidably disposed within the outer shell.

15. The clip applier according to claim 14, wherein the plunger assembly of the at least one adapter assembly includes a stem and a plunger slidable relative to the stem, the plunger including a head and a shaft extending distally from the head.

16. The clip applier according to claim 15, wherein the plunger assembly further includes a biasing member disposed about the stem and the shaft of the plunger and positioned between the collar and the head of the plunger, the plunger assembly configured such that slidable insertion of the shaft of the plunger into the stem compresses the biasing member.

17. The clip applier according to claim 15, wherein distal movement of the plunger of the handle assembly urges the plunger of the at least one adapter assembly distally to actuate the clip cartridge assembly, end effector, or surgical tool associated with that adapter assembly.

18. A method of assembling a reposable endoscopic surgical clip applier, comprising:
inserting a plunger assembly into a proximal portion of a lumen defined within a barrel of a housing, the housing further including a stationary handle supporting the barrel thereon;
engaging a plug within the proximal portion of the lumen of the barrel to close off the proximal portion of the lumen of the barrel and retain the plunger assembly therein;
inserting an adapter assembly into a distal portion of the lumen of the barrel such that the adapter assembly is operably positioned relative to the plunger assembly; and
engaging the adapter assembly within the distal portion of the lumen of the barrel.

19. The method according to claim 18, further including, prior to engaging the plug, moving a gripping portion of a trigger operably associated with the housing towards the stationary handle such that a stem portion of the trigger abuts a proximal end of the plunger assembly.

20. The method according to claim 18, wherein engaging the adapter assembly within the distal portion of the lumen of the barrel includes rotating a lock collar relative to both the adapter assembly and the barrel.
